(19) United States
(12) Patent Application Publication
(10) Pub. No.: US 2019/0254892 A1
(43) Pub. Date: Aug. 22, 2019

(54) SELF ADHESIVE BANDAGE ROLL DISPENSER SYSTEM

(71) Applicants: Matthew Klatman, Levittown, NY (US); Sara Vitale, Levittown, NY (US)

(72) Inventors: Matthew Klatman, Levittown, NY (US); Sara Vitale, Levittown, NY (US)

(21) Appl. No.: 16/281,005

(22) Filed: Feb. 20, 2019

Related U.S. Application Data
(60) Provisional application No. 62/632,534, filed on Feb. 20, 2018.

(51) Int. Cl. A61F 15/00; B65H 16/00; B65H 37/00

(52) U.S. Cl. CPC A61F 15/002; B65H 37/005; B65H 16/005; A61F 15/005

(57) ABSTRACT

A self adhesive bandage roll dispenser system including a dispenser unit having a dispensing aperture, a prong assembly, a wheel mechanism, and a control button, a series of gauze bandages configured in a roll each including, an adhesive-side, a non-adhesive-side, a first-end, and a second-end. The roll comprises a series of individual gauze bandages connected at perforated sections. The gauze bandages are dispensed through the dispensing aperture and may be removed at the perforated sections. The prong assembly removes paper layering on the gauze bandages for ready application. The present invention provides users with an easily-accessible first aid tool capable of dispensing and applying gauze bandages to a wound.

### CROSS REFERENCE TO RELATED APPLICATION

The present application is related to and claims priority to U.S. Provisional Patent Application No. 62/632,534 filed Feb. 20, 2018, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of dispensers and more specifically relates to roll dispensers.

### 2. Description of Related Art

Open wounds are consistently at risk for infection. People often apply gauze bandages to an open wound, keeping the affected area protected from contracting germs and bacteria. If infection occurs, the wound may not heal properly and continue to reopen. Loose bandage application may require constant replacement in order to keep the wound protected. Standard bandages are often stored in small boxes or first aid kits and finding a specific size of bandage can be difficult and frustrating. A suitable solution is desired.

U.S. Pat. No. 6,213,343 to Gerry Damikolas relates to a portable sterile bandage dispenser. The described portable sterile bandage dispenser is for dispensing bandages from a roll of sterilized, individually wrapped bandages. Several bandage feeding mechanisms are disclosed, to serially dispense a bandage and simultaneously strip off the liner surrounding the bandage, upon depression of a hand actuated switch. Also disclosed is an improved roll of bandages suitable for use in the bandage dispenser.

### BRIEF SUMMARY OF THE INVENTION

In view of the foregoing disadvantages inherent in the known dispenser art, the present disclosure provides a novel self adhesive bandage roll dispenser system. The general purpose of the present disclosure, which will be described subsequently in greater detail, is to provide a portable device for dispensing gauze bandages.

A self adhesive bandage roll dispenser system is disclosed herein. The self adhesive bandage roll dispenser system includes a dispenser unit including a dispensing aperture, a prong assembly, a wheel mechanism, and a control button, a series of gauze bandages configured in a roll each including, an adhesive-side, a non-adhesive-side, a first-end, and a second-end. The dispenser unit houses the roll of the gauze bandages within. The roll comprises a series of individual gauze bandages connected at perforated sections and forming a continuous the roll. The roll of the gauze bandages are supported on the wheel mechanism which is configured to rotate and feed the gauze bandages through the dispensing aperture. The adhesive-side comprises a gauze section positioned between the first-end and second-end of each the gauze bandage. The adhesive-side comprises a removable covering. The prong assembly includes two teeth-like members configured to remove the removable covering as the series of gauze bandages are dispensed. The teeth-like members are positioned to provide a channel therebetween for the series of gauze bandages to pass therethrough while the removable covering is removed. The prong assembly is positioned at the dispensing aperture. The control button is a finger-activated trigger for ejecting of the gauze bandages individually.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a self adhesive bandage roll dispenser system, constructed and operative according to the teachings of the present disclosure.

FIG. 1 is a perspective view of the self adhesive bandage roll dispenser system, according to an embodiment of the disclosure.

FIG. 2 is a perspective view of the self adhesive bandage roll dispenser system of FIG. 1, according to an embodiment of the present disclosure.

FIG. 3 is a perspective view of the self adhesive bandage roll dispenser system of FIG. 1, according to an embodiment of the present disclosure.

FIG. 4 is a perspective view of the self adhesive bandage roll dispenser system of FIG. 1, according to an embodiment of the present disclosure.

FIG. 5 is a perspective view of the self adhesive bandage roll dispenser system of FIG. 1, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to dispensers and more particularly to a self adhesive bandage roll dispenser system as used to improve the efficient dispensing of gauze bandages for medical purposes.

Generally, the present invention provides users with an easily-accessible first aid tool capable of dispensing and applying gauze bandages to a wound. It includes a button or switch which will initiate the bandage out of the opening where teeth-like prongs peel the protective backing off the bandage, effectively preparing the bandage for application. The device features perforations located on each bandage, offering a simple way for the tool to separate the bandages for independent application until an upwards motion is performed, tearing off at the closest perforation. It effectively eliminates the need to constantly locate individual bandages should a wound need to be protected, allowing users to store the device in a purse, backpack, pocket, glove compartment, office desk, first aid kit, and many other locations. Further, it allows professionals in the medical field, such as nurses and doctors, to hold the bandage applicator in scrub pockets for easy access. The present invention enables children to quickly and easily apply their own bandages.

The self adhesive bandage roll dispenser is a small plastic tool designed to apply individual bandages to open wounds on any part of the body. The device is portable and is comprised of a round dispenser unit with a roll of perforated gauze bandages stored within. A user friendly grip allows for convenient access to a switch or button used to dispense the stored bandages. The bandages can be wrapped around a wheel mechanism capable of being actuated and dispensing the bandages. A mechanism can feed out the bandage tip and plastic teeth will be used to peel off the protective covering on the adhesive. As pressure is applied, the wheel turns from the tension and the bandage is forced through the front opening. At the end of application, users can move the dispenser up or down to tear off the bandage from its perforation. The bandage is applied over an affected area for protecting the wound. Self adhesive bandage roll dispenser can be constructed using plastic and other suitable materials. The device is intended to be relatively small in size, capable of fitting in a purse, backpack, pocket, etc. Exact size, measurement, construction, and design specifications may vary upon manufacturing.

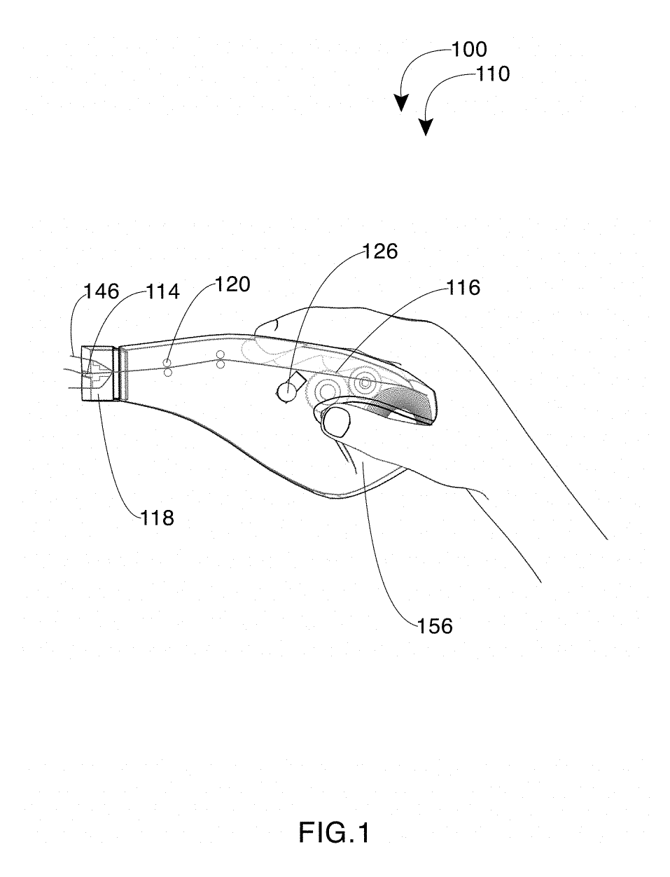

Referring now more specifically to the drawings by numerals of reference, there is shown in FIGS. 1-5, various views of a self adhesive bandage roll dispenser system 100. FIG. 1 shows a self adhesive bandage roll dispenser system 100, according to an embodiment of the present disclosure. As illustrated, the self adhesive bandage roll dispenser system 100 may include a dispenser unit 110 including a dispensing aperture 114, a prong assembly 118, a wheel mechanism 122, and a control button 126, a series of gauze bandages 130 configured in a roll each including, an adhesive-side 132, a non-adhesive-side 134, a first-end 136, and a second-end 138. As designed, the device offers a simple and convenient tool for applying gauze bandages 130.

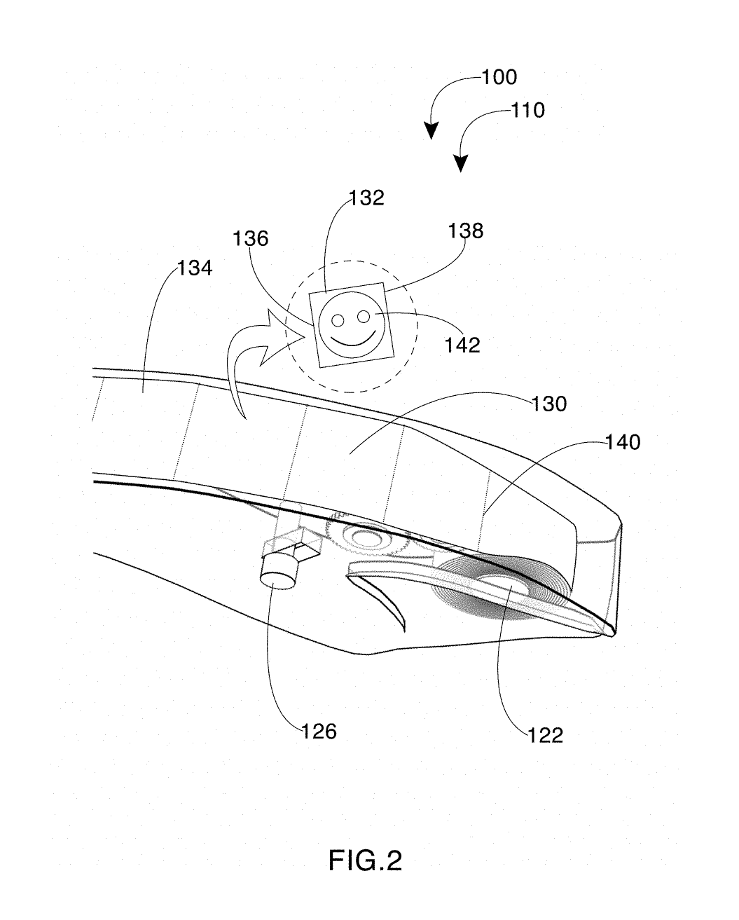

FIG. 2 shows a perspective view of the self adhesive bandage roll dispenser system 100 of FIG. 1, according to an embodiment of the present disclosure. As above, the self adhesive bandage roll dispenser system 100 may include the dispenser unit 110 and the series of gauze bandages 130 configured in a roll housed within the dispenser unit 110 and configured to be ejected through the dispensing aperture 114. The dispenser unit 110 houses the roll of the gauze bandages 130 within. The roll comprises a series of individual gauze bandages 130 connected at perforated sections 140 and forming a continuous the roll. The roll of the gauze bandages 130 are supported on the wheel mechanism 122 and the wheel mechanism 122 is configured to rotate and feed the gauze bandages 130 through the dispensing aperture 114. The adhesive-side 132 comprises a gauze section 142 positioned between the first-end 136 and second-end 138 of each the gauze bandage 130. The adhesive-side 132 comprises a removable covering 146. The prong assembly 118 is configured to remove the removable covering 146 as the series of gauze bandages 130 are dispensed through the dispensing aperture 114. The control button 126 is to activate dispensing and ejecting of the gauze bandages 130.

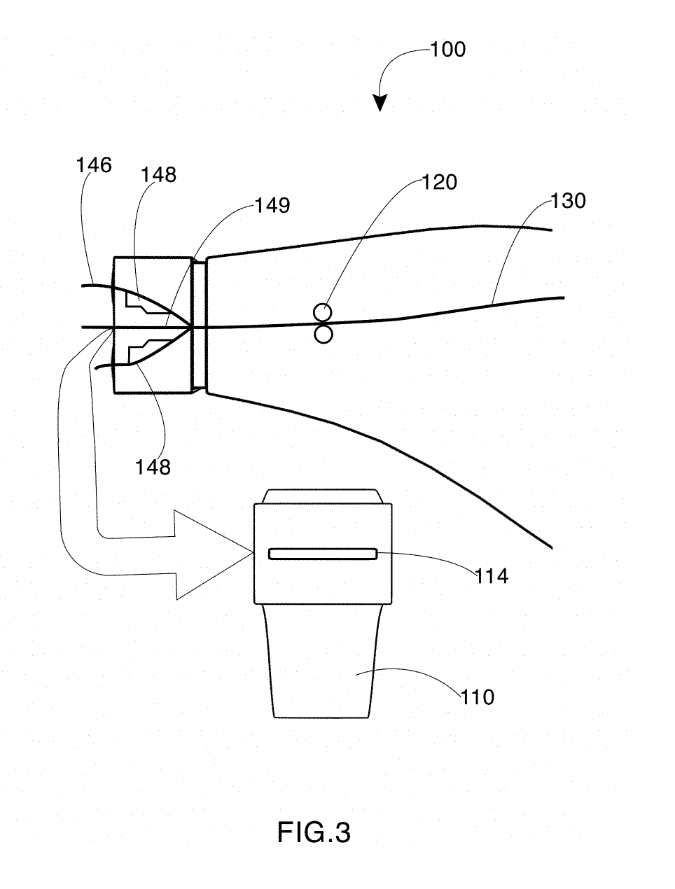

FIG. 3 shows a perspective view of the self adhesive bandage roll dispenser system 100 of FIG. 1, according to an embodiment of the present disclosure. As above, the self adhesive bandage roll dispenser system 100 may include the dispenser unit 110 including the dispensing aperture 114, the prong assembly 118, the wheel mechanism 122, and the control button 126, the series of gauze bandages 130 configured in a roll each including, an adhesive-side 132, a non-adhesive-side 134, a first-end 136, and a second-end 138. The series of gauze bandages 130 are each perforated at the first-end 136 and the second-end 138 such that a user may remove each one of the gauze bandages 130 individually as dispensed. The gauze bandages 130 are separable from one another at the perforated sections 140. Adhesive sections surround the gauze section 142 on the adhesive-side 132. The gauze bandages 130 are self-adhesive-bandages. The gauze bandages 130 comprise cotton gauze bandages suitable for treatment of medical wounds.

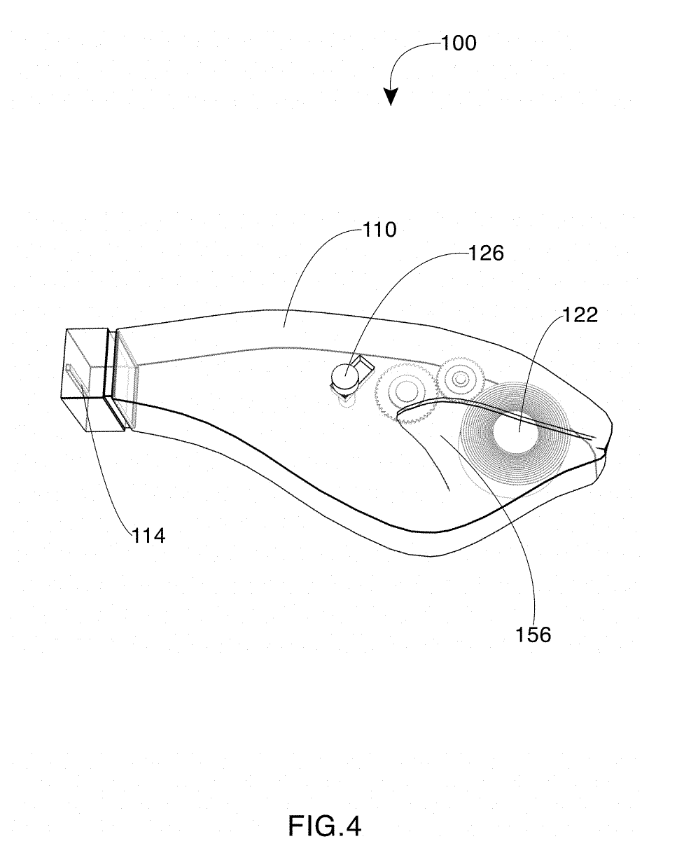

FIG. 4 shows a perspective view of the self adhesive bandage roll dispenser system 100 of FIG. 1, according to an embodiment of the present disclosure. As above, the self adhesive bandage roll dispenser system 100 may include the dispenser unit 110 including the dispensing aperture 114, the prong assembly 118, the wheel mechanism 122, and the control button 126. The control button 126 is a finger-activated trigger for ejecting of the gauze bandages 130 individually. The prong assembly 118 includes two teeth-like members 148 configured to remove the removable covering 146 as the series of gauze bandages 130 are dispensed. The teeth-like members 148 are positioned to provide a channel 149 therebetween for the series of gauze bandages 130 to pass therethrough while the removable covering 146 is removed. The prong assembly 118 is positioned at the dispensing aperture 114. The prong assembly 118 removes paper layering from the series of gauze bandages 130.

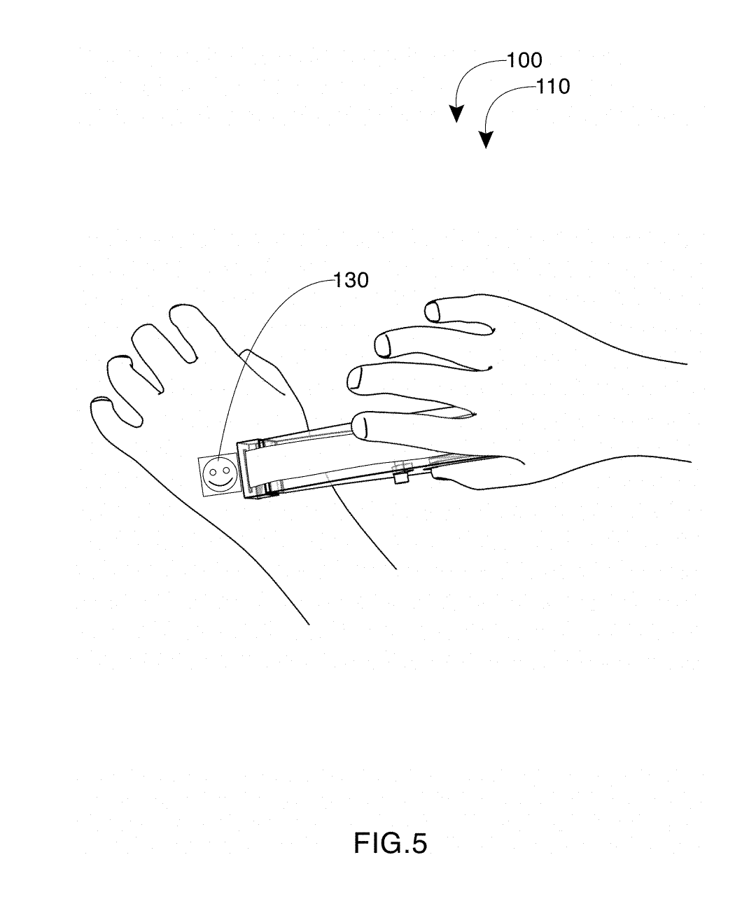

FIG. 5 shows a perspective view of the self adhesive bandage roll dispenser system 100 of FIG. 1, according to an embodiment of the present disclosure. As above, the self adhesive bandage roll dispenser system 100 may include the dispenser unit 110 housing the series of gauze bandages 130 configured in a roll. When a tension force is applied to one of the gauze bandages 130 extending through the dispensing aperture 114, the gauze bandages 130 are dispensed therethrough and applied to a surface. The dispenser unit 110 houses a single the roll of the series of gauze bandages 130. The dispenser unit 110 preferably comprises an ergonomic hand grip 156 which contours to a profile of a hand of a user. The dispenser unit 110 further includes a feeder mechanism 120 and a gear assembly 116. The feeder mechanism 120 comprises a series of guiding members for feeding the series of gauze bandages 130 toward the dispensing aperture 114.

According to one embodiment, the self adhesive bandage roll dispenser system 100 may be arranged as a kit 105. In particular, the self adhesive bandage roll dispenser system 100 may further include a roll of a series of gauze bandages 130; a dispenser unit 110 and a set of instructions. The instructions may detail functional relationships in relation to the structure of the self adhesive bandage roll dispenser system 100 such that the self adhesive bandage roll dispenser system 100 can be used, maintained, or the like, in a preferred manner.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A self adhesive bandage roll dispenser system comprising:
a dispenser unit including;
a dispensing aperture;
a prong assembly;
a wheel mechanism; and
a control button;
a series of gauze bandages configured in a roll each including;
an adhesive-side;
a non-adhesive-side;
a first-end; and
a second-end;
wherein said dispenser unit houses said roll of said gauze bandages within;
wherein said roll comprises a series of individual gauze bandages connected at perforated sections and forming a continuous said roll;
wherein said roll of said gauze bandages are supported on said wheel mechanism, said wheel mechanism is configured to rotate and feed said gauze bandages through said dispensing aperture;
wherein said adhesive-side comprises a gauze section positioned between said first-end and second-end of each said gauze bandage;
wherein said adhesive-side comprises a removable covering, said prong assembly is configured to remove said removable covering as said series of gauze bandages are dispensed through said dispensing aperture; and
wherein said control button is to activate dispensing and ejecting of said gauze bandages.

2. The self adhesive bandage roll dispenser system of claim 1, wherein said series of gauze bandages are each perforated at said first-end and said second-end.

3. The self adhesive bandage roll dispenser system of claim 2, wherein said gauze bandages are separable from one another at said perforated sections.

4. The self adhesive bandage roll dispenser system of claim 3, wherein adhesive sections surround said gauze section on said adhesive-side.

5. The self adhesive bandage roll dispenser system of claim 4, wherein said gauze bandages are self-adhesive-bandages.

6. The self adhesive bandage roll dispenser system of claim 1, wherein said control button is a finger-activated trigger for ejecting of said gauze bandages individually.

7. The self adhesive bandage roll dispenser system of claim 1, wherein said prong assembly includes two teeth-like members configured to remove said removable covering as said series of gauze bandages are dispensed.

8. The self adhesive bandage roll dispenser system of claim 7, wherein said teeth-like members are positioned to provide a channel therebetween for said series of gauze bandages to pass therethrough while said removable covering is removed.

9. The self adhesive bandage roll dispenser system of claim 8, wherein said prong assembly is positioned at said dispensing aperture.

10. The self adhesive bandage roll dispenser system of claim 7, wherein said prong assembly removes paper layering from said series of gauze bandages.

11. The self adhesive bandage roll dispenser system of claim 1, wherein when a tension force is applied to one of said gauze bandages extending through said dispensing aperture, said gauze bandages are dispensed therethrough.

12. The self adhesive bandage roll dispenser system of claim 1, wherein said gauze bandages comprise cotton gauze bandages suitable for treatment of medical wounds.

13. The self adhesive bandage roll dispenser system of claim 1, wherein said dispenser unit houses a single said roll of said series of gauze bandages.

14. The self adhesive bandage roll dispenser system of claim 1, wherein said dispenser unit further comprises an ergonomic hand grip.

15. The self adhesive bandage roll dispenser system of claim 14, wherein said ergonomic hand grip contours to a profile of a hand of a user.

16. The self adhesive bandage roll dispenser system of claim 1, wherein said dispenser unit further includes a feeder mechanism.

17. The self adhesive bandage roll dispenser system of claim 1, wherein said feeder mechanism comprises a series of guiding members for feeding said series of gauze bandages toward said dispensing aperture.

18. The self adhesive bandage roll dispenser system of claim 1, wherein said dispenser unit further comprises a gear assembly.

19. A self adhesive bandage roll dispenser system, the self adhesive bandage roll dispenser system comprising:
a dispenser unit including;
a dispensing aperture;
a prong assembly;
a wheel mechanism; and
a control button;
a series of gauze bandages configured in a roll each including;
an adhesive-side;
a non-adhesive-side;
a first-end; and
a second-end;
wherein said dispenser unit houses said roll of said gauze bandages within;
wherein said roll comprises a series of individual gauze bandages connected at perforated sections and forming a continuous said roll;
wherein said roll of said gauze bandages are supported on said wheel mechanism, said wheel mechanism is configured to rotate and feed said gauze bandages through said dispensing aperture;
wherein said adhesive-side comprises a gauze section positioned between said first-end and second-end of each said gauze bandage;
wherein said adhesive-side comprises a removable covering, said prong assembly is configured to remove said removable covering as said series of gauze bandages are dispensed through said dispensing aperture;
wherein said control button is to activate dispensing and ejecting of said gauze bandages;
wherein said series of gauze bandages are each perforated at said first-end and said second-end;
wherein said gauze bandages are separable from one another at said perforated sections;
wherein adhesive sections surround said gauze section on said adhesive-side;
wherein said gauze bandages are self-adhesive-bandages;
wherein said control button is a finger-activated trigger for ejecting of said gauze bandages individually;
wherein said prong assembly includes two teeth-like members configured to remove said removable covering as said series of gauze bandages are dispensed;
wherein said teeth-like members are positioned to provide a channel therebetween for said series of gauze bandages to pass therethrough while said removable covering is removed;
wherein said prong assembly is positioned at said dispensing aperture;
wherein said prong assembly removes paper layering from said series of gauze bandages;
wherein when a tension force is applied to one of said gauze bandages extending through said dispensing aperture, said gauze bandages are dispensed therethrough;
wherein said gauze bandages comprise cotton gauze bandages suitable for treatment of medical wounds;
wherein said dispenser unit houses a single said roll of said series of gauze bandages;
wherein said dispenser unit further comprises an ergonomic hand grip;
wherein said ergonomic hand grip contours to a profile of a hand of a user;
wherein said dispenser unit further includes a feeder mechanism;
wherein said feeder mechanism comprises a series of guiding members for feeding said series of gauze bandages toward said dispensing aperture; and
wherein said dispenser unit further comprises a gear assembly.

20. The self adhesive bandage roll dispenser system of claim 19, further comprising set of instructions; a roll of a series of gauze bandages; a dispenser unit and
wherein the self adhesive bandage roll dispenser system is arranged as a kit.
